(19) United States
(12) Patent Application Publication
(10) Pub. No.: US 2008/0065192 A1
(43) Pub. Date: Mar. 13, 2008

(54) Compliance Graded Stent

(75) Inventor: Joseph Berglund, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/531,316

(22) Filed: Sep. 13, 2006

(51) Int. Cl. A61F 2/06

(52) U.S. Cl. 623/1.15

(57) ABSTRACT

In one embodiment, an intraluminal stent includes a stent framework with a first end portion, a second end portion, and a center portion includes a plurality of struts positioned between the first end portion and the second end portion. The first end portion includes a plurality of struts and the second end portion includes a plurality of struts. The first end portion plurality of struts and second portion plurality of struts have a radial strength and/or stiffness less than a radial strength and/or stiffness of the center portion plurality of struts. In another embodiment, a method of treating a vascular condition includes delivering a stent to a target region of a vessel via a catheter. The stent is deployed at the target region. The first and second end portions of the deployed stent are flexed in a radial direction while reducing flexing in the radial direction of the center portion.

### TECHNICAL FIELD OF THE INVENTION

The present invention relates generally to the field of intraluminal medical devices. More particularly, the invention relates to an intraluminal stent, an intraluminal stent delivery system, and method of manufacturing an intraluminal stent.

### BACKGROUND OF THE INVENTION

Coronary artery disease (CAD) results from arteriosclerosis of blood vessels serving the heart. Arteriosclerosis is a hardening and narrowing of the arteries commonly accompanied by a deposition of waxy substance therein. This substance, known as plaque, is made of cholesterol, fatty compounds, calcium, and the blood-clotting material fibrin. Often the arteries of the heart can suddenly become so severely blocked that there is an inadequate blood supply after the blockage, leading to the occurrence of a myocardial infarction or “heart attack.” Although some heart attacks are caused by such “hard” plaques, many are caused by “soft” or vulnerable plaques. A vulnerable plaque is an inflamed part of an artery that can burst. This can lead to the formation of a blood clot, which can reduce or block the flow of blood.

Soon after a myocardial infarction, the area of cardiac tissue downstream of the blockage may suffer damage. The damage is caused by a lack of adequate blood flow, known as ischemia, as the tissue is starved of oxygen and nutrients. Unless the blockage is resolved relatively quickly, the ischemic cells begin to die. Often, a surgical procedure, such as a Coronary Artery By-Pass Grafting (CABG), is used to graft new blood vessels to the ischemic area to improve circulation. Alternatively, a Percutaneous Transluminal Coronary Angioplasty (PTCA) procedure oftentimes accompanied by stenting of the blocked vessel is performed to reopen the vessel and maintain blood flow. However, by-passing or reopening of the arteries is sometimes not possible or at least not immediately possible because of limitations of present methodologies, risk to the patient from surgical intervention, or other circumstances.

Plain-old-balloon-angioplasty (POBA) is an exemplary medical procedure to widen obstructed blood vessels narrowed by plaque deposits. The procedure may be used in coronary or peripheral arteries. In an angioplasty procedure, a catheter having a special inflatable balloon on its distal end is navigated through the patient's arteries and is advanced through the artery to be treated to position the balloon within the narrowed region (stenosis). The region of the stenosis is expanded by inflating the balloon under pressure to forcibly widen the artery. After the artery has been widened, the balloon is deflated and the catheter is removed from the patient.

A significant difficulty associated with balloon angioplasty is that in a considerable number of cases the artery may again become obstructed in the same region where the balloon angioplasty had been performed. The repeat obstruction may be immediate (abrupt reclosure), which is usually caused by an intimal flap or a segment of plaque or plaque-laden tissue that loosens or breaks free as a result of the damage done to the arterial wall during the balloon angioplasty. Such abrupt reclosure may block the artery requiring emergency surgery. This risk also necessitates the presence of a surgical team ready to perform such emergency surgery when performing balloon angioplasty procedures. More commonly, closure of the artery (restenosis) may occur later, for example, two or more months after the angioplasty for reasons not fully understood and may require repeat balloon angioplasty or bypass surgery. When such longer-term restenosis occurs, it usually is more similar to the original stenosis, that is, it is in the form of cell proliferation and renewed plaque deposition in and on the arterial wall.

To reduce the incidence of re-obstruction and restenosis, several strategies have been developed. Implantable devices, such as stents, have been used to reduce the rate of angioplasty related re-obstruction and restenosis by about half. The use of stent devices has greatly improved the prognosis of the patients. The stent is placed inside the blood vessel after the angioplasty has been performed. A catheter typically is used to deliver the stent to the arterial site to be treated. The stent may further include one or more therapeutic substance(s) impregnated or coated thereon to limit re-obstruction and/or restenosis.

One shortcoming of certain current stent designs relates to the fact that the end portions of the stent are generally rigid in nature, much like the center portion of the stent. For example, stents manufactured from metals (e.g., stainless steel, cobalt chromium, nitinol, etc.) exhibit negligible stretch (e.g., compression and expansion) during pulsatile blood flow. The stents are rigid to resist compressive forces (i.e., caused by restenosis) of the artery along its entire length. Unlike the stent, most arteries are relatively flexible wherein arteries exhibit about a 10 percent stretch in their diameter during pulsatile blood flow. The rigidity of certain stents near their end portions may lead to abrupt changes in mechanical compliance which could lead to chronic irritation, abnormal hemodynamic blood flow and arterial damage. What is desirable, then, is a stent that resists restenosis and includes end portions that are more compliant with the arterial wall.

Accordingly, it would be desirable to provide a stent with a compliance gradient to overcome the aforementioned and other limitations.

### SUMMARY OF THE INVENTION

A first aspect according to the invention provides an intraluminal stent. The stent includes a stent framework with a first end portion, a second end portion, and a center portion includes a plurality of struts positioned between the first end portion and the second end portion. The first end portion includes a plurality of struts and the second end portion includes a plurality of struts. The first end portion plurality of struts and second portion plurality of struts have a radial stiffness less than a radial stiffness of the center portion plurality of struts.

A second aspect according to the invention provides an intraluminal stent delivery system. The system includes a catheter and a stent framework with a first end portion, a second end portion, and a center portion includes a plurality of struts positioned between the first end portion and the second end portion. The first end portion includes a plurality of struts and the second end portion includes a plurality of struts. The first end portion plurality of struts and second portion plurality of struts have a radial stiffness less than a radial stiffness of the center portion plurality of struts.

A third aspect according to the invention provides a method of deploying an intraluminal stent. The method includes delivering a stent to a target region of a vessel via a catheter. The stent is deployed at the target region. The stent includes a first end portion, a second end portion, and a center portion disposed between the first and second end portions. The first and second end portions of the deployed stent are flexed in a radial direction while reducing flexing in the radial direction of the center portion.

The foregoing and other features and advantages of the invention will become further apparent from the following description of the presently preferred embodiments, read in conjunction with the accompanying drawings. The drawings have not been drawn to scale. The detailed description and drawings are merely illustrative of the invention, rather than limiting the scope of the invention being defined by the appended claims and equivalents thereof.

### BRIEF DESCRIPTION OF THE DRAWINGS

FIG. 1 illustrates a stent delivery system in accordance with the present invention;

FIG. 2 illustrates a detailed view of one embodiment of a stent positioned in a blood vessel shown with compressed end portions, in accordance with the present invention;

FIG. 3 illustrates a detailed view of the stent shown in FIG. 2 with expanded end portions;

FIG. 4 illustrates a first embodiment of an alternative strut configuration, in accordance with the present invention;

FIG. 5 illustrates a second embodiment of an alternative strut configuration, in accordance with the present invention;

FIG. 6 illustrates a third embodiment of an alternative strut configuration, in accordance with the present invention;

FIG. 7 illustrates a first embodiment of alternative strut materials, in accordance with the present invention; and

FIG. 8 illustrates a flowchart of a method of deploying an intraluminal stent in accordance with the present invention.

### DESCRIPTION OF THE PRESENTLY PREFERRED EMBODIMENTS

The following description relates primarily to the positioning and operation of an intravascular stent for treating an ischemic coronary artery of a patient after a myocardial infarction. The treatment may occur, for example, before, during, and/or after a CABG or PTCA procedure in an effort to salvage and/or rehabilitate myocardial tissue. Those skilled in the art will recognize that although the present invention is described primarily in the context of localized delivery of a stent in a coronary blood vessel with a specific intravascular device, the Inventors contemplate numerous other applications of a prosthetic device in accordance with said invention.

For example, an intravascular stent according to the present invention may be deployed within another arteriole or venous blood vessel, or adapted as an intraluminal device for use in another vessel such as the intestine, air duct, esophagus, bile duct, and the like. Any number of devices capable of performing the prescribed method(s) may be adapted for use with the present invention. Furthermore, the deployment strategies, treatment site and tissues, and therapeutic agents are not limited to those described. Numerous modifications, substitutions, additions, and variations may be made to the devices and methods while providing a stent in accordance with the present invention.

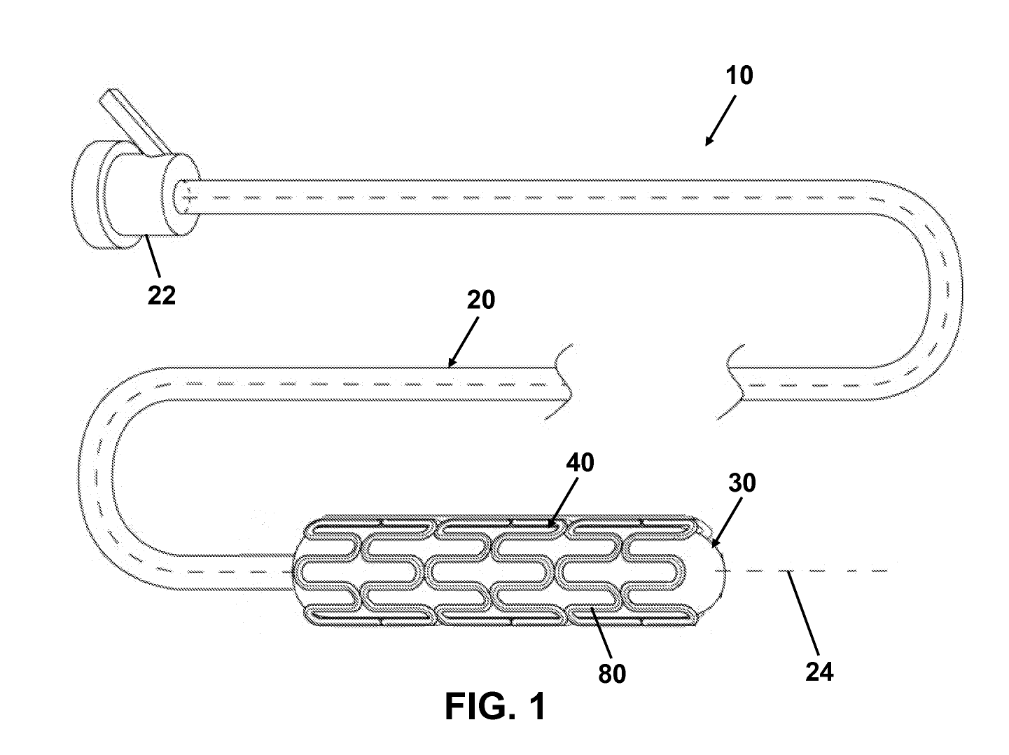

Referring to the drawings, wherein like reference numerals refer to like elements, FIG. 1 is a perspective view of an intraluminal stent delivery system, in accordance with one embodiment of the present invention and shown generally by numeral 10. System 10 includes a catheter 20, a balloon 30 operably attached to the catheter 20, and a stent 40 disposed on the balloon 30. Stent 40 remains compressed on the balloon 30 during advancement through the vasculature. The compressed stent 40 includes a small profile (i.e., cross-sectional size). In another embodiment, a sheath may be disposed on the stent 40 to protect the stent 40 as well as the vessel walls during advancement. Balloon 30 and stent 40 are shown in an expanded (deployed) configuration.

In one embodiment, the catheter 20 may comprise an elongated tubular member manufactured from one or more polymeric materials, sometimes in combination with metallic reinforcement. In some applications (such as smaller, more tortuous arteries), it is desirable to construct the catheter from very flexible materials to facilitate advancement into intricate access locations. Numerous over-the-wire, rapid-exchange, and other catheter designs are known and may be adapted for use with the present invention. Catheter 20 may be secured at its proximal end to a suitable Luer fitting 22. Catheter 20 may be manufactured from a material such as a thermoplastic elastomer, urethane, polymer, polypropylene, plastic, ethelene chlorotrifluoroethylene (ECTFE), polytetrafluoroethylene (PTFE), fluorinated ethylene propylene copolymer (FEP), nylon, Pebax® resin, Vestamid® nylon, Tecoflex® resin, Halar® resin, Hyflon® resin, Pellathane® resin, combinations thereof, and the like. Catheter 20 may include an aperture formed at a distal rounded end allowing advancement over a guidewire 24.

In one embodiment, the stent 40 embodying features of the invention can be readily delivered to a desired body lumen, such as a coronary artery (peripheral vessels, bile ducts, etc.), by mounting the stent 40 on an expandable member of a delivery catheter, for example the balloon 30, and advancing the catheter 20 and stent assembly through the body lumen to a target site. Generally, the stent 40 is compressed or crimped onto the balloon 30 portion of the catheter 20 so that the stent 40 does not move longitudinally relative to the balloon 30 portion of the catheter 20 during delivery through the arteries, and during expansion of the stent 40 at the target site. In another embodiment, the stent may be manufactured from a resilient material and expand at the target site after it is properly positioned. During the deployment process, for example, a sheath enclosing a crimped stent may be withdrawn thereby allowing the stent to expand outwardly into contact with the vessel wall. Typically, self-expanding stents do not require a balloon.

Balloon 30 may be any variety of balloons capable of expanding the stent 40. Balloon 30 may be manufactured from any sufficiently elastic material such as polyethylene, polyethylene terephthalate (PET), nylon, or the like. Stent 40 may be expanded with the balloon 30. System 10 may optionally include a sheath (not shown) to retain the stent 40 in a collapsed state and to prevent contact with surfaces, such as a vessel wall, during advancement through a vessel lumen and subsequent deployment. Once the stent 40 is properly positioned, the sheath may be retracted thereby allowing the stent to assume its expanded shape. In addition, once the stent 40 is properly positioned within the vasculature, the balloon 30 and stent 40 are expanded together. Balloon 30 may then be deflated and retracted thereby allowing the stent 40 to remain in a deployed configuration. Alternatively, for self-expanding stents balloon or other expandable members are typically not used. Instead, a sheath covering the compressed stent may be withdrawn (at the treatment site) thereby allowing the stent to expand to its naturally larger shape into contact with the vessel. The advancement, positioning, and deployment of stents and like devices are well known in the art. In addition, those skilled in the art will recognize that numerous devices and methodologies may be adapted for deploying the stent in accordance with the present invention.

The terms “catheter” and “stent”, as used herein, may include any number of intravascular and/or implantable prosthetic devices (e.g., a stent-graft); the examples provided herein are not intended to represent the entire myriad of devices that may be adapted for use with the present invention. Although the devices described herein are primarily done so in the context of deployment within a blood vessel, it should be appreciated that intravascular and/or implantable prosthetic devices in accordance with the present invention may be deployed in other vessels, such as a bile duct, intestinal tract, esophagus, airway, etc. Further, the terms “biodegradable” and “non-biodegradable”, as used herein, refer to a relative stabilities of substances when positioned within a living being. For example, a biodegradable substance will degrade (i.e., break down) at a faster rate than a non-biodegradable substance. A non-biodegradable substance, however, may, eventually degrade given a sufficient amount of time.

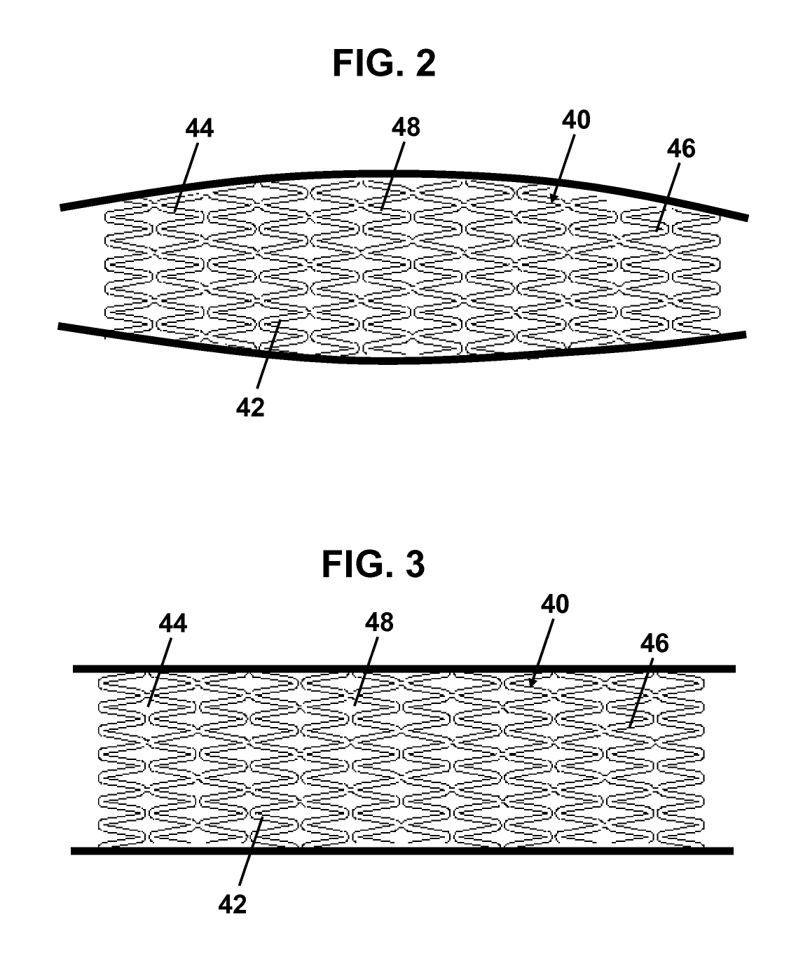

Referring to FIGS. 2 and 3, FIG. 2 illustrates one embodiment of a compliance-graded stent in a compressed configuration and FIG. 3 illustrates the compliance-graded stent in an expanded configuration. In one embodiment, the stent 40 includes a frame 42 with a first end portion 44, a second end portion 46, and a center portion 48 positioned in between the first and second end portions 44, 46. A radial stiffness of the first end portion 44 is less than a radial stiffness of the center portion 48. A radial stiffness of the second end portion 46 is less than a radial stiffness of the center portion 48. The greater radial stiffness of the center portion 48 relative to the first and second end portions 44, 46 provides a compliance-graded stent 40 (i.e., lower axial force resistance at the first and second end portions 44, 46 in comparison to the center portion 48). The first and second end portions 44, 46 compress in a radial direction, as shown in FIG. 2, and expand, as shown in FIG. 3, during pulsatile flow of the blood vessel (i.e., thereby mimicking the vessel). Meanwhile, the center portion 48 of the stent 40 remains relatively stiff (e.g., uncompressed) thereby maintaining the openness of the blood vessel.

In one embodiment, at least one of the first and second end portions 44, 46 include an alternative strut configuration in comparison to the center portion 48. As such, the first and second end portions 44, 46 match the compliance of the blood vessel.

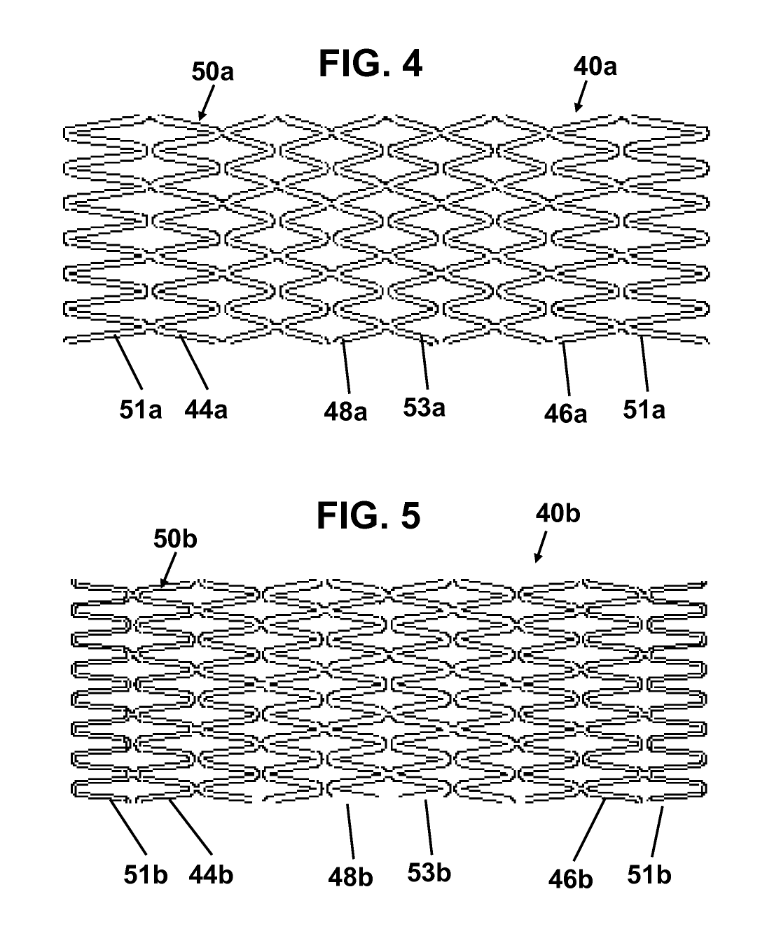

In one embodiment of the alternative strut configuration, as shown in FIG. 4, the stent 40a includes struts 50a that are of modified strut density. In one embodiment, for example, struts 51a at first and second end portions 44a, 46a are longer than struts 53a at center portion 48a; thereby bending moments applied to the crowns are increased and radial stiffness of the struts is decreased at first and second end portions 44a, 46a in comparison with the center portion 48a (i.e., modified strut density). One skilled in the art can appreciate that a number of strut configurations may provide modified strut radial stiffness and is not limited to the embodiment provided herein.

In another embodiment of alternative strut configuration, as shown in FIG. 5, the stent 40b includes struts 50b that are of modified strut size (e.g., width). In one embodiment, struts 51b of the first and second end portions 44b, 46b have a width that is narrower than the width of struts 53b that comprise center portion 48b. In one embodiment, the width of struts 51b located at the first and second end portions are about one half the width of struts 53b located in the center portion.

In another embodiment of alternative strut configuration, the thickness of the struts located in the first and second end portions 44b, 46b are substantially less than the thickness of the struts located in the center portion 48b of stent 40b. In one embodiment, for example, struts 51b at first and second end portions 44b, 46b are relatively thinner in comparison to struts 53b at center portion 48b. In one embodiment, struts 51b are about one half the thicknesses of struts 53b. One skilled in the art can appreciate that a number of strut configurations may provide a modified strut width and is not limited to the embodiment provided herein.

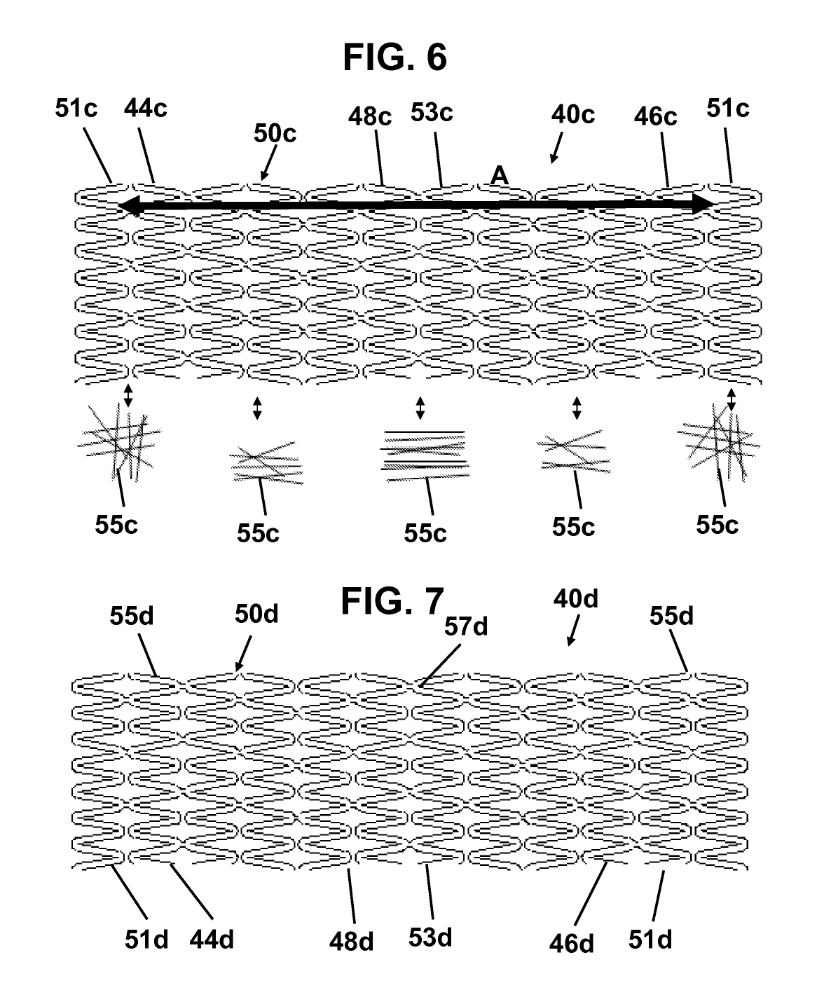

In yet another embodiment of alternative strut configuration, as shown in FIG. 6, the stent 40c includes struts 50c that possess modified material alignment. For example, materials in struts 55c at first and second end portions 44c, 46c are relatively misaligned in comparison to materials in struts 53c at the center portion 48c. Misalignment may be achieved by, for example, providing a polymeric (e.g., biodegradable) stent that includes polymer fibers, which are shown in detail below the stent 40c in corresponding sections, that aligned differently along the stent 40c axis A. In this case, fibers 55c positioned at first and second end portions 44c, 46c are relatively misaligned (e.g., such as a random orientation) whereas the fibers 55c become closer to a parallel alignment (i.e., unidirectional) in an axial direction toward the center portion 48c of the stent. As appreciated by one skilled in the art, a parallel arrangement of fibers 55c enhances strength of a material to forces exerted perpendicular to said fibers 55c. Therefore, the stent 40 may include microfibers 44 arranged substantially parallel to (i.e., in an axial direction) to the vessel wall thereby providing additional resistance to forces acting to crimp the stent 40 shut (i.e., forces generated during restenosis). A substantially parallel arrangement is used in, for example, laminated materials (e.g., plywood) wherein the material is much stronger across its grain than parallel to it. Polymer may be one or more polymers known in the art for use of prosthetic devices such as stents. Some exemplary polymers that may be adapted for use with the present invention include, but are not limited to, polycaprolactone, polylactide, polyglycolide, polyorthoesters, polyanhydrides, poly(amides), poly(alkyl-2-cyanocrylates), poly(dihydropyrans), poly(acetals), poly(phosphazenes), poly(dioxinones), trimethylene carbonate, polyhydroxybutyrate, polyhydroxyvalerate, their copolymers, blends, and copolymer blends, combinations thereof, and the like. Fiber alignment can be accomplished using constituents of the polymeric stent or by incorporating additional reinforcement components. Reinforcement fibers can be manufactured from various materials known in the art including, but not limited to, carbon fiber and Kevlar® synthetic fiber. One skilled in the art can appreciate that a number of strut configurations may provide an alternative strut configuration and is not limited to the embodiment provided herein.

In one embodiment, at least one of the first and second end portions 44, 46 include alternative strut materials from the center portion. As such, the first and second end portions 44, 46 match the compliance of the blood vessel.

In one embodiment of alternative strut materials, as shown in FIG. 7, the stent 40d includes struts 50d that are manufactured from graded flexible materials. For example, struts 51d at first and second end portions 44d, 46d are manufactured from a relatively more flexible material 55d (i.e., in terms of resisting compressive forces) in comparison to material 57d of struts 53d at center portion 48d. In another embodiment, three or more materials may be used to make up the gradient. In yet another embodiment, one material that is modified so as to produce different species of the material having different degrees of flexibility may be used to make up the gradient. For example, relatively stiff material(s) (i.e. MP35N or SS316L) may be used in the center portion 48d of the stent, while different, relatively less stiff material(s) (i.e. Nitinol or Mg WE43), may be used in the end portions 44d, 46d. One skilled in the art can appreciate that a number of material configurations may provide mechanical gradients and is not limited to the embodiments provided herein.

In one embodiment, at least one of the first and second end portions 44, 46 of the stent 40 include an alternative strut processing condition from the center portion 48. As defined herein, an alternative strut processing condition refers to one or more chemical or physical processes applied to the stent 40 material(s) of the first and/or second end portions 44, 46 as compared to the center portion 48.

In one embodiment of an alternative strut processing condition, a polymeric stent 40 includes edges that are annealed at the first and second end portions 44d, 46d. Specifically, the first and second end portions 44d, 46d are heated and then cooled quickly to remove polymer crystallinity in the stent 40 material thereby increasing the flexibility of the constituent material. One skilled in the art will recognize an annealing process may be applied along various degrees to the first and second end portions 44d, 46d. For example, the first and second end portions 44d, 46d may be annealed to the same extent or at a gradually decreasing level from the edges toward the center portion 48d.

In another embodiment of an alternative strut processing condition, a metallic stent 40 includes a middle segment 48c that has been cold-worked through processes including swaging or rolling. In another embodiment of an alternative strut processing conditions, a cold-worked metallic stent 40 includes end portions 44d, 46d that have been annealed at elevated temperatures to reduce dislocation densities in the material. One skilled in the art will recognize an annealing process may be applied along various degrees to the first and second end portions 44d, 46d. For example, the first and second end portions 44d, 46d may be annealed to the same extent or at a gradually decreasing level from the edges toward the center portion 48d.

Those skilled in the art will appreciate that the compliance-graded stent 40 is not limited to the alternative strut configuration, alternative strut materials, and alternative strut processing condition embodiment provided herein. Numerous other strategies are contemplated by the Inventor for providing a compliant stent and fall within the spirit and scope of the present invention.

In one embodiment, as shown in FIG. 1, the stent includes at least one therapeutic agent 80 coated on a surface of the stent 40. Therapeutic agent 80 may be a gene therapy agent or a drug agent such as an antiangiogenesis agent, antiarteriosclerotic agent, antiarythmic agent, antibiotic, antibody, anticoagulant, antidiabetic agent, antiendothelin agent, antihypertensive agent, antiinflammatory agent, antimitogenic factors, antineoplastic agent, antioxidants, antiplatelet agent, antipolymerases, antiproliferative agent, antirestenotic drug, antisense agent, antithrombogenic agent, calcium channel blockers, chemotherapeutic agent, clot dissolving agent, fibrinolytic agent, growth factor, growth factor inhibitor, immunosuppressant, nitrate, nitric oxide releasing agent, remodeling inhibitors, vasodilator, agent having a desirable therapeutic application, and the like. Specific examples of gene therapy agents include a recombinant DNA product, a recombinant RNA product, stem cells, engineered or altered cells, and a virus mediated gene therapy agent. Specific example of drugs include abciximab, angiopeptin, calcium channel blockers, colchicine, eptifibatide, heparin, hirudin, lovastatin, methotrexate, streptokinase, taxol, ticlopidine, tissue plasminogen activator, steroid, trapidil, urokinase, vasodilators, vasospasm inhibitors, and growth factors (e.g., VEGF, TGF-beta, IGF, PDGF, and FGF). In another or the same embodiment, the therapeutic 80 agent may be substance(s) that reduce tissue ischemia. This may be necessary in instances when surgical intervention is not immediately possible to remove a myocardial infarction.

In one embodiment, the therapeutic agent may additionally include one or more polymers, solvents, a component thereof, a combination thereof, and the like. For example, the therapeutic agent may include a mixture of a gene therapy agent/drug and a polymer dissolved in a compatible liquid solvent as known in the art. Polymer(s) provide a matrix for incorporating the gene therapy agent/drug within a coating and, optionally, provide means for slowing the elution of an underlying therapeutic agent when it comprises a cap coat. Some exemplary biodegradable polymers that may be adapted for use with the present invention include, but are not limited to, polycaprolactone, polylactide, polyglycolide, polyorthoesters, polyanhydrides, poly(amides), poly(alkyl-2-cyanocrylates), poly(dihydropyrans), poly(acetals), poly(phosphazenes), poly(dioxinones), trimethylene carbonate, polyhydroxybutyrate, polyhydroxyvalerate, their copolymers, blends, and copolymers blends, combinations thereof, and the like.

Solvents are used to dissolve the therapeutic agent(s), gene therapy agent(s), and polymer(s) to provide a therapeutic agent coating solution. Some exemplary solvents that may be adapted for use with the present invention include, but are not limited to, acetone, ethyl acetate, tetrahydrofuran (THF), chloroform, N-methylpyrrolidone (NMP), methylene chloride, and the like.

Those skilled in the art will recognize that the nature of the gene therapy agent, drug, and polymer may vary greatly and are typically formulated to achieve a given therapeutic effect, such as limiting restenosis, thrombus formation, hyperplasia, etc. Once formulated, a therapeutic agent solution (mixture) comprising the coating may be applied to the stent 40 by any of numerous strategies known in the art including, but not limited to, spraying, dipping, rolling, nozzle injection, and the like. Numerous strategies of applying the coating in accordance with the present invention are known in the art.

In one embodiment, two or more therapeutic agents are incorporated into the stent 40 and are released having a multiple elution profile. For example, a first therapeutic agent disposed on the stent 40 is released to reduce inflammation. The first agent may be released on a short-term basis to overcome surgical trauma of the treatment. A second therapeutic agent may be disposed underneath the first therapeutic agent on the stent 40 for reducing endovascular restenosis. After the first therapeutic agent has been delivered, the second therapeutic agent is released on a longer-term basis.

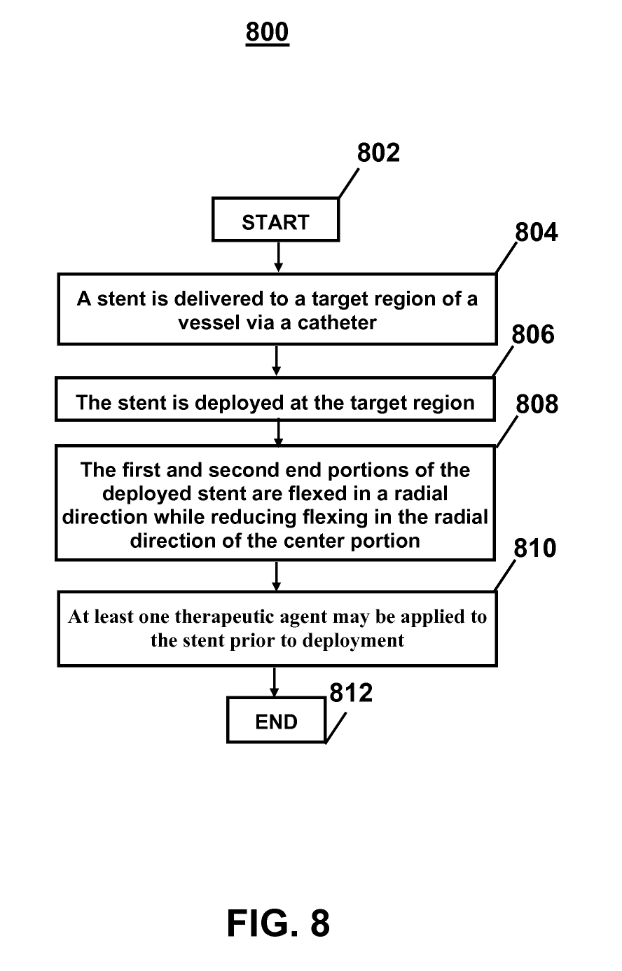

FIG. 8 is a flowchart illustrating method 800 of deploying an intraluminal stent, in accordance with the present invention. The method begins at step 802. In one embodiment, a stent is delivered to a target region of a vessel via a catheter (step 804). The stent is deployed at the target region (step 806). The stent includes a first end portion, a second end portion, and a center portion disposed between the first and second end portions. The first and second end portions of the deployed stent are able to flex in a radial direction while the center portion (step 808) possesses reduced flexibility in the radial direction. In another or the same embodiment, a portion or the entirety of the stent may be biodegradable.

At step 810, at least one therapeutic agent may be applied to the stent 40 prior to deployment. Numerous processes are known in the art for applying the therapeutic agent to the stent 40. Once formulated, a therapeutic agent (mixture) comprising the coating(s) may be applied to the stent by any of numerous strategies known in the art including, but not limited to, spraying, dipping, rolling, nozzle injection, and the like. It will be recognized that the at least one therapeutic agent coating may be alternatively layered, arranged, configured on/within the stent depending on the desired effect (i.e., The coatings may be positioned on various portions of the stent 40). Before application, one or more primers may be applied to the stent to facilitate adhesion of the at least one therapeutic agent coating. Numerous strategies of applying the primer(s), therapeutic agent coating(s), and cap coat(s) in accordance with the present invention are known in the art. Various drug elution profiles may be achieved by differentially coating/impregnating the therapeutic agent(s) within the polymeric structure and/or on the stent as understood by one skilled in the art. Specifically, those skilled in the art will recognize that the nature of the drugs, polymers, and solvent may vary greatly and are typically formulated to achieve a given therapeutic effect, such as limiting restenosis, thrombus formation, hyperplasia, etc. Once formulated, a therapeutic agent (mixture) comprising the coating(s) may be applied to the stent by any of numerous strategies known in the art including, but not limited to, spraying, dipping, rolling, nozzle injection, and the like, or, alternatively, added to the polymer of the stent during manufacture. It will be recognized that the at least one therapeutic agent coating may be alternatively layered, arranged, configured on/within the stent depending on the desired effect. Before application, one or more primers may be applied to the stent to facilitate adhesion of the at least one therapeutic agent coating. Once the at least one therapeutic agent coating is/are applied, it/they may be dried (i.e., by allowing the solvent to evaporate) and, optionally, other coating(s) (e.g., a “cap” coat) added thereon. Numerous strategies of applying the primer(s), therapeutic agent coating(s), and cap coat(s) in accordance with the present invention are known in the art.

The method may end at step 812 and be repeated as necessary.

While the embodiments of the invention disclosed herein are presently considered to be preferred, various changes and modifications may be made without departing from the spirit and scope of the invention. The intraluminal stent delivery system, stent, and method of deploying the stent of the present invention are not limited to any particular design, configuration, methodology, or sequence. For example, the catheter, stent, frame, first end portion, second end portion, and center portion may vary without limiting the utility of the invention. Furthermore, the described order of the method may vary and may include additional steps to manufacture the stent.

Upon reading the specification and reviewing the drawings hereof, it will become immediately obvious to those skilled in the art that myriad other embodiments of the present invention are possible, and that such embodiments are contemplated and fall within the scope of the presently claimed invention. The scope of the invention is indicated in the appended claims, and all changes that come within the meaning and range of equivalents are intended to be embraced therein.

1. An intraluminal stent comprising:
a stent framework comprising a first end portion, a second end portion, and a center portion having a plurality of struts positioned between the first end portion and the second end portion; the first end portion having a plurality of struts and the second end portion having a plurality of struts; wherein the first end portion plurality of struts and second end portion plurality of struts have a radial stiffness and/or strength less than a radial stiffness and/or strength of the center portion plurality of struts.

2. The intraluminal stent of claim 1 comprising one or more intermediate portions between the end and center portions wherein the intermediate portions have a radial stiffness and/or strength between the radial stiffness and/or strength of the end and center portions.

3. The intraluminal stent of claim 1 wherein the radial stiffness and/or strength between the end and center portions increases as a gradient such that a continuum between the portions is obtained.

4. The intraluminal stent of claim 1 wherein at least one portion of the stent framework is biodegradable.

5. The intraluminal stent of claim 1 wherein at least one of the first end portion and the second end portion comprises an alternative strut configuration in comparison to the center portion.

6. The intraluminal stent of claim 5 wherein the alternative strut configuration is selected from a group consisting of modified strut density, modified strut size, and modified strut alignment.

7. The intraluminal stent of claim 1 wherein at least one of the first and second end portions comprise alternative strut materials from the center portion.

8. The intraluminal stent of claim 7 wherein the alternative strut materials comprises strut material compositions.

9. The intraluminal stent of claim 1 wherein at least one of the first and second end portions comprise an alternative strut processing condition from the center portion.

10. The intraluminal stent of claim 9 wherein the alternative strut processing condition is selected from a group consisting of annealing stent edges and aligning edge material.

11. The intraluminal stent of claim 1 further comprising at least one therapeutic agent disposed on the frame.

12. An intraluminal stent delivery system comprising:
a catheter; and
a stent framework comprising at least a first end portion, a second end portion, and a center portion having a plurality of struts positioned between the first end portion and the second end portion; the first end portion having a plurality of struts and the second end portion having a plurality of struts; wherein the first end portion plurality of struts and second portion plurality of struts have a radial stiffness and/or strength less than a radial stiffness and/or strength of the center portion plurality of struts.

13. The system of claim 12 wherein a portion of the stent framework is biodegradable.

14. The system of claim 12 wherein at least one of the first end portion and the second end portions comprise an alternative strut configuration in comparison to the center portion.

15. The system of claim 14 wherein the alternative strut configuration is selected from a group consisting of modified strut density, modified strut size, and modified strut alignment.

16. The system of claim 12 wherein at least one of the first and second end portions comprise alternative strut materials from the center portion.

17. The system of claim 16 wherein the alternative strut materials comprises graded flexible materials.

18. The system of claim 12 wherein at least one of the first and second end portions comprise an alternative strut processing condition from the center portion.

19. The system of claim 18 wherein the alternative strut processing condition is selected from a group consisting of annealing stent edges and aligning edge material.

20. The system of claim 12 further comprising at least one therapeutic agent disposed on the frame.

21. A method of treating a vascular condition, the method comprising:
delivering a stent to a target region of a vessel via a catheter;
deploying the stent at the target region, the stent including at least a first end portion, a second end portion, and a center portion disposed between the first and second end portions; and
flexing the first and second end portions of the deployed stent in a radial direction while reducing flexing in the radial direction of the center portion.

22. The method of claim 21 wherein the stent is biodegradable.

23. The method of claim 21 wherein the center portion of the stent comprises a stent framework including a first plurality of struts and the first and the second end portions of the stent comprise a stent framework including a second plurality of struts.

24. The method of claim 21 wherein at least a portion of the first plurality of struts have a first density and at least a portion of the second plurality of struts have a second density, wherein the first density is greater than the second density.

25. The method of claim 21 wherein at least a portion of the first plurality of struts have a first size and at least a portion of the second plurality of struts have a second size, wherein the first size is greater than the second size.

26. The method of claim 21 wherein at least a portion of the second plurality of struts comprise a modified strut alignment.

27. The method of claim 21 wherein at least a portion of the second plurality of struts comprise graded flexible materials.

28. The method of claim 21 wherein at least a portion of the second plurality of struts comprise annealed stent edges.

29. The method of claim 21 wherein at least a portion of the second plurality of struts comprise aligned edge material.

30. The method of claim 21 further comprising providing at least one therapeutic agent disposed on the stent.
